(19) United States
(12) Patent Application Publication
(10) Pub. No.: US 2004/0009963 A1
(43) Pub. Date: Jan. 15, 2004

(54) Use of salmeterol and fluticasone propionate combination

(76) Inventors: Donald Herbert Horstman, Research Triangle Park, NC (US); Claire Julia Maden, Research Triangle Park, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/363,438

(22) PCT Filed: Aug. 31, 2001
(86) PCT No.: PCT/GB01/03928

(30) Foreign Application Priority Data
Aug. 31, 2000 (US) 60229381

(51) Int. Cl. A61K 31/56; A61K 31/137

(52) U.S. Cl. 514/171; 514/651

(57) ABSTRACT

The present invention relates to the use of salmeterol and fluticasone propionate combinations for the treatment of chronic obstructive pulmonary disease.

The present invention relates to the use of salmeterol and fluticasone propionate combinations for the treatment of chronic obstructive pulmonary disease.

The combination of the beta-2 adrenergic agonist salmeterol or a physiologically acceptable salt thereof and the corticosteroid fluticasone propionate has been described in GB 2 235 627 for use in the treatment of asthma and other respiratory disorders.

Fluticasone propionate is itself known from GB 2 088 877 to have anti-inflammatory activity and to be useful for the treatment of allergic and inflammatory conditions of the nose, throat, or lungs such as asthma and rhinitis, including hay fever. However, the clinical utility of inhaled corticosteroids in the treatment of chronic obstructive pulmonary disease is uncertain as discussed, for example, in the editorials by Calverley and Bames, American Journal of Respiratory and Critical Care Medicine, vol 161, pp341-344, 2000.

Salmeterol is known from GB 2 140 800 and is used clinically in the form of its xinafoate salt for the treatment of asthma and chronic obstructive pulmonary disease.

Chronic obstructive pulmonary disease (COPD) is a general term encompassing chronic bronchitis, emphysema, and chronic obstructive airways disease. COPD is a chronic slowly progressive disorder characterised by airways obstruction which does not change markedly over several months. Unlike asthma, airflow limitation in COPD as measured by FEV, (forced expiratory volume) can never be returned to normal values. Symptoms of COPD, which vary with the severity of the disease, include coughing with or without sputum, and breathlessness (dyspnea) with or without wheezing. In the UK during the period 1990 to 1992 there were 81,500 deaths attributable to COPD in people aged over 65. There still exists the need for further therapeutic agents which could be used in the clinical management of COPD.

In a more particular aspect, the present invention relates to treatment and alleviation of the symptoms associated with COPD, particularly of breathlessness, to improvement in health status and to a reduction in exacerbation rate including those requiring treatment with oral corticosteroids.

We now propose that the use of a combination of salmeterol or a physiologically acceptable salt thereof and fluticasone propionate may have clinical advantages in the treatment of COPD over the use of salmeterol alone.

Accordingly, the present invention provides a method for treatment of COPD in a mammal, such as a human, which comprises administering an effective amount of a combination of salmeterol or a physiologically acceptable salt thereof, such as the xinafoate salt, and fluticasone propionate.

In the alternative, there is provided the use of a combination of salmeterol or a physiologically acceptable salt thereof, such as the xinafoate salt, and fluticasone propionate for the manufacture of a medicament for treatment of COPD.

As used herein, the term “treatment” means the improvement of clinical outcome, for example, improvement of lung function and/or alleviation of symptoms such as breathlessness (dyspnea) with or without wheezing, and/or improvement in health status, and/or a reduction in exacerbation rate including those requiring treatment with oral corticosteroids. Health status may be measured using the St. George's Respiratory Questionnaire (Jones P W, Quirk F H, Baveystock C M, and Littlejohns P., A self-complete measure of health status for chronic airflow limitation. The St George's Respiratory Questionnaire, Am. Rev. Respir. Dis., vol. 145, pp 1321-7, 1992).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

It will be appreciated that the compounds of the salmeterol and fluticasone propionate combination may be administered simultaneously, either in the same or different pharmaceutical formulations, or sequentially. Where there is sequential administration, the delay in administering the second and any subsequent active ingredient should not be such as to lose the beneficial therapeutic effect of the combination of the active ingredients. In a preferred aspect of the invention, the salmeterol or its physiologically acceptable salt and the fluticasone propionate are administered as a combined pharmaceutical formulation. The weight/weight ratio of salmeterol to fluticasone administered according to the invention is preferably in the range 4:1 to 1:20.

The amount of salmeterol or a physiologically acceptable salt thereof, such as the xinafoate salt, and fluticasone propionate which is required to achieve a therapeutic effect will, of course, vary with the particular salt form, the route of administration, the subject under treatment, and the particular disorder or disease being treated. The combination of the invention may be administered by inhalation to an adult human at a dose of from 50 μg to 2000 μg per day, suitably 100 μg to 1500 μg per day, more suitably 500 μg to 1000 μg per day of fluticasone propionate and 50 μg to 200 μg per day, suitably 50 μg to 100 μg per day of salmeterol. Preferred combinations include 250 μg or 500 μg of fluticasone propionate and 50 μg of salmeterol. The daily dose may be administered as several sub-doses, for example, twice daily.

While it is possible for salmeterol or a physiologically acceptable salt thereof, such as the xinafoate salt, and fluticasone propionate to be administered as raw drugs, it is preferable to present each of them as a pharmaceutical formulation.

Hereinafter, the term “active ingredient” means salmeterol or a physiologically acceptable salt thereof, such as the xinafoate salt, and/or fluticasone propionate.

Suitable formulations include those suitable for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Preferably, the pharmaceutical formulations used in accordance with the present invention are suitable for administration by inhalation. Inhalation formulations may be in the form of powder compositions which will preferably contain lactose, or spray compositions which may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluoroethane, carbon dioxide or other suitable gas. Suitable aerosol spray compositions for use in accordance with the invention are described in WO 93/11743.

A preferred formulation is a powder composition comprising salmeterol or a physiologically acceptable salt thereof, such as the xinafoate salt, fluticasone propionate and lactose.

Another preferred formulation is an aerosol formulation consisting of salmeterol or a physiologically acceptable salt thereof, such as the xinafoate salt, fluticasone propionate, and 1,1,1,2,3,3,3-heptafluoropropane and/or 1,1,1,2-tetrafluoroethane as propellant.

Capsules and cartridges of for example gelatin, or blisters of for example laminated aluminium foil, for use in an inhaler or insuflator may be formulated containing a powder mix of a compound of the invention and a suitable powder base such as lactose or starch.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

It should be understood that in addition to the ingredients particularly mentioned above, the formulations used according to the invention may include other agents conventional in the art having regard to the type of formulation in question, for example those suitable for oral administration may include flavouring agents. Furthermore, the combination of salmeterol or a physiologically acceptable salt thereof, such as the xinafoate salt, and fluticasone propionate used according to the present invention may be used in combination with a further active ingredient, for example another bronchodilator suitably an anticholinergic such as ipratropium, tiotropium, or oxitropium, or a methylxanthine such as theophylline, another anti-inflammatory drug such as sodium cromoglycate or nedocromil sodium, an antihistamine or mucolytic.

Thus according to a further aspect of the invention, there is provided a pharmaceutical formulation for the treatment of COPD comprising salmeterol or a physiologically acceptable salt thereof, such as the xinafoate salt, and fluticasone propionate, and a pharmaceutically acceptable carrier or excipient, and optionally one or more other therapeutic agents. Preferably, the pharmaceutical formulation is in a form which is suitable for inhalation.

### EXAMPLES

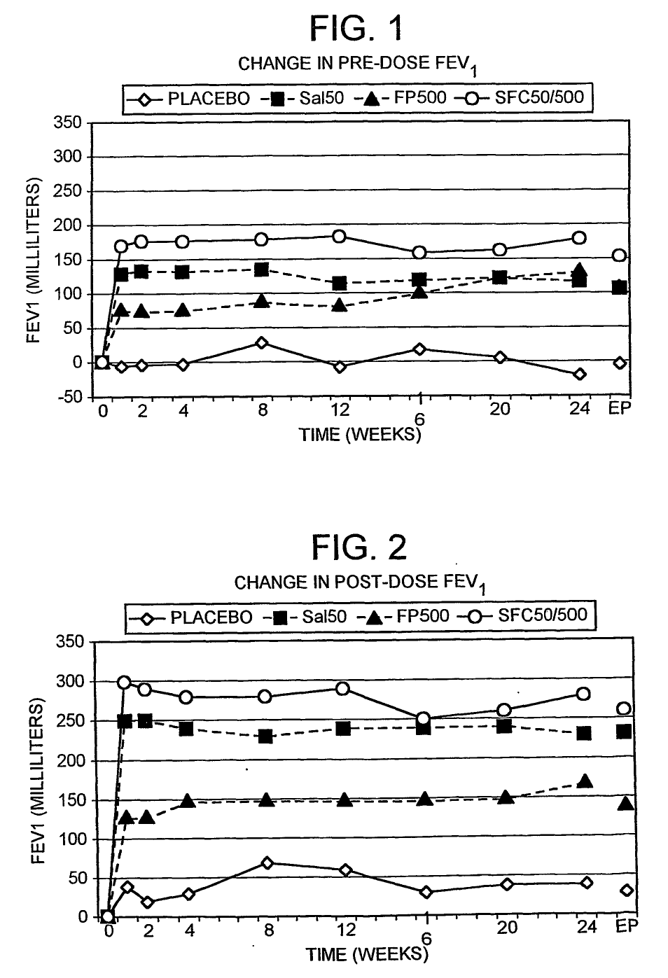

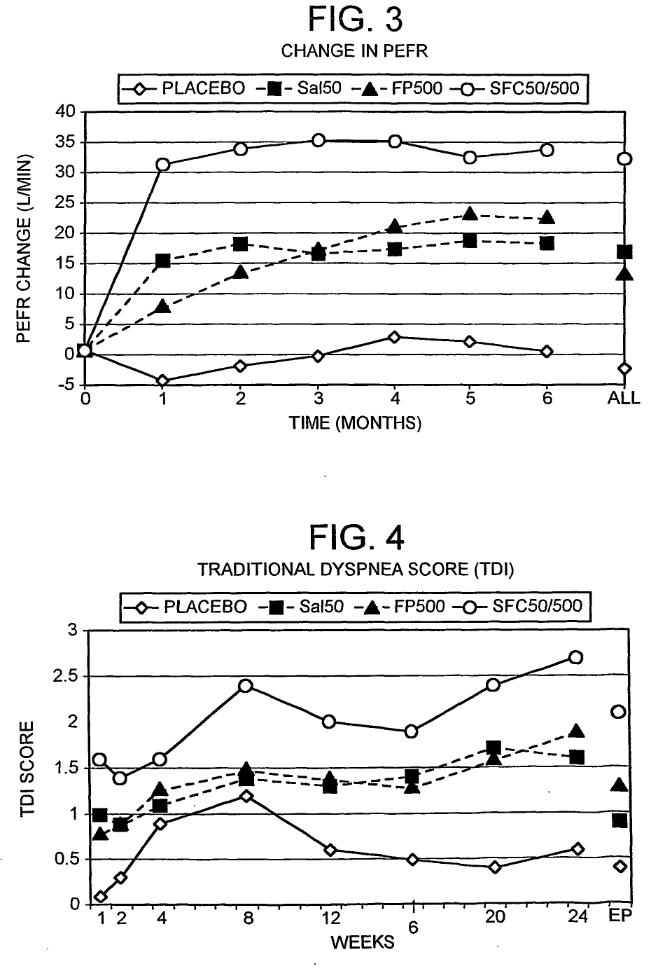

A randomised, double-blind, parallel group 6 month clinical trial was conducted to compare the effects of an inhaled salmeterol and fluticasone propionate combination product, inhaled salmeterol, inhaled fluticasone propionate, and placebo in COPD patients. Each group of patients was treated with either salmeterol/fluticasone propionate 50 μg/500 μg (165 patients), salmeterol 50 μg (160 patients), fluticasone propionate 500 μg (168 patients), or placebo (181 patients), all administered twice daily by a dry-powder inhaler (DISKUS™, Glaxo Wellcome). Salmeterol, both as part of the combination product and as a monotherapy, was administered as its xinafoate salt. 71% of the patients included in the study met the poorly reversible criteria for COPD, i.e. ΔFEV, less than 10% predicted following inhalation of 400 μg salbutamol (a short-acting beta-2 agonist). The differences between the predose FEV₁ on the first day of treatment and predose FEV₁ at subsequent treatment visits was measured, the results of which are shown in FIG. 1. Post-dose FEV, and PEFR were measured similarly giving the results shown in FIGS. 2 and 3 respectively. The Transitional Dyspnea Score (TDI) (see Mahler D A, Weinberg D H, Wells C K, and Feinstein A R; Chest, (1984) 85:751-8) was also measured at each visit and the results are shown in FIG. 4.

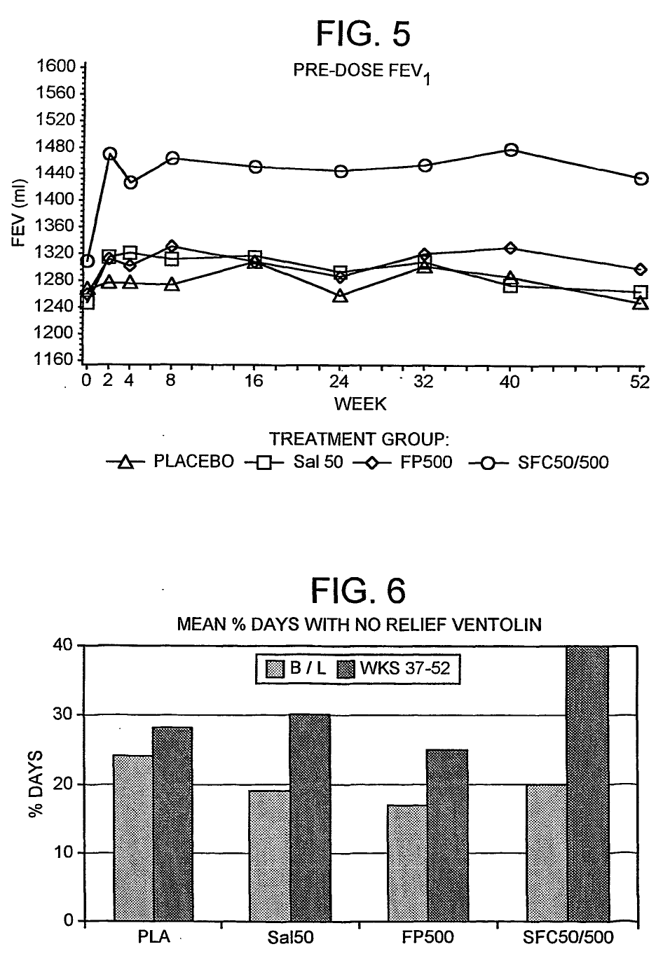

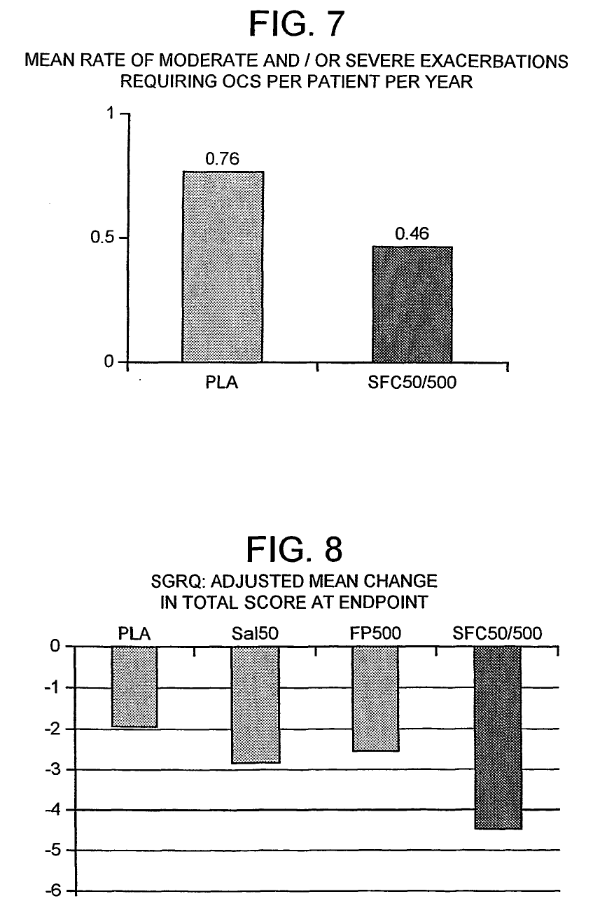

In a further placebo-controlled clinical trial of 12 months treatment duration in patients with COPD, regular use of an inhaled salmeterol xinafoate and fluticasone propionate combination product rapidly improved lung function, reduced breathlessness and reduced the use of relief medication. FIG. 5 shows the mean improvement in pre-dose FEV₁ over time for all the patients enrolled in the trial (the intent-to-treat population). FIG. 6 shows the mean % days when no relief medication (Ventolin™ (salbutamol), Glaxo Wellcome) was required. In addition the risk of COPD exacerbation and the need for additional courses of oral corticosteroids was significantly reduced, as shown in FIG. 7. There were also significant improvements in health status, as measured using the St. George's Respiratory Questionnaire (Jones P W, Quirk F H, Baveystock C M, and Littlejohns P., A self-complete measure of health status for chronic airflow limitation. The St George's Respiratory Questionnaire, Am. Rev. Respir. Dis., vol. 145, pp 1321-7, 1992), and shown in FIG. 8.

In FIGS. 1 to 8, the abbreviations used are as follows:

Sal50: patients receiving salmeterol 50 μg

FP500: patients receiving fluticasone propionate 500 μg

SFC50/500: patients receiving salmeterol/fluticasone propionate 50 μg/500 μg.

FEV₁: forced expiratory volume in one second

PEFR: peak expiratory flow rate

EP: end point

PLA: placebo

OCS: oral corticosteroid

B/L: baseline (prior to commencement of trial)

SGQR: St George's Respiratory Questionnaire

wks: weeks into trial

1. A method for treatment of COPD in a mammal, such as a human, which comprises administering an effective amount of a combination of salmeterol or a physiologically acceptable salt thereof and fluticasone propionate.

2. A method according to claim 1 wherein the salmeterol or physiologically acceptable salt thereof and fluticasone propionate are administered as a combined pharmaceutical formulation.

3. A method according to claim 1 or 2 in which the salmeterol or physiologically acceptable salt thereof and fluticasone propionate are administered by inhalation.

4. A method according to any one of claims 1 to 3 in which the salmeterol is administered as the xinafoate salt.

5. Use of a combination of salmeterol or a physiologically acceptable salt thereof and fluticasone propionate for the manufacture of a medicament for the treatment of COPD.

6. Use according to claim 5 wherein the medicament is a combined pharmaceutical formulation.

7. Use according to claim 5 or 6 in which the medicament is suitable for inhalation therapy.

8. Use according to any one of claims 5 to 7 in which the salmeterol is in the form of the xinafoate salt.

9. A pharmaceutical formulation for the treatment of COPD comprising salmeterol or a physiologically acceptable salt thereof and fluticasone propionate, and a pharmaceutically acceptable carrier or excipient and optionally one or more other therapeutic agents.

10. A pharmaceutical formulation according to claim 9 which is in a form suitable for inhalation.

11. A pharmaceutical formulation according to either claim 9 or 10 in which the salmeterol is in the form of the xinafoate salt.
